(19) United States
(12) Patent Application Publication
(10) Pub. No.: US 2015/0223825 A1
(43) Pub. Date: Aug. 13, 2015

(54) DISPOSABLE MEDICAL DRILL GUIDE

(71) Applicant: ECA Medical Instruments, Newbury Park, CA (US)

(72) Inventors: John Nino, Simi Valley, CA (US); Douglas Slomski, Chicago, IL (US)

(73) Assignee: ECA Medical Instruments, Newbury Park, CA (US)

(21) Appl. No.: 14/693,830

(22) Filed: Apr. 22, 2015

Related U.S. Application Data
(63) Continuation-in-part of application No. 14/000,741, filed on Oct. 23, 2013.
(60) Provisional application No. 61/464,118, filed on Feb. 28, 2011.

(51) Int. Cl. A61B 17/17

(52) U.S. Cl. CPC A61B 17/17; A61B 2017/0023

(57) ABSTRACT

A drill guide which is disposable and may be packaged in a kit with varying drill guides and sleeve guides is disclosed. Said device may have medical waste collection indentations, regions, or wells, whereby medical waste is removed and disposed of along with said device.

This patent application is a Continuation-In-Part of Ser. No. 14/000,741 filed Oct. 23, 2013, which is a National Stage of International patent application PCT/US2012/026756, filed Feb. 27, 2012, which claims the full Paris Convention benefit of and priority to U.S. Provisional Patent Application Ser. No. 61/464,118 filed Feb. 28, 2011, the contents of which are incorporated by reference herein in their entirety, as if fully set forth herein.

### BACKGROUND

1. Field

This disclosure relates to a disposable drill guide.

2. General Background

Traditional medical drill guides require sterilization and are susceptible to coating or caking with medical waste. Any medical waste they become contaminated with must be removed. Medical waste may make the handle slippery. When drilling medical waste is generated.

### SUMMARY

Human bone, tissue, blood and other fluids are frequently present during surgical procedures. These materials may be medical waste. Medical waste includes: “Any discarded biologic product such as blood or tissue removed from operating rooms, morgues, laboratories, or other medical facilities. The term may also be applied to bedding, bandages, syringes, and similar materials that have been used in treating patients and to animal carcasses or body parts used in research. Medical waste is regulated at the state and local levels. Mosby's Medical Dictionary, 8th edition. © 2009, Elsevier.

Medical waste is regulated and needs to be properly disposed of Part of the disposal process is the collection of such medical waste.

The wells and/or collection zones on the drill guide handle in proximity with the interface of drill handle with user's hand can channel medical waste off all of the handle surfaces and direct said waste to areas of the handle and in some instances areas of the handle that are removed from the area of the handle (the outer silhouette) that the user grasps and also reduces slippage.

When properly gloved the gloved hand of a user will pick-up or be covered with medical waste it is exposed to during medical procedures. The well structures or zones of the handle are transfer or collection points to acquire, capture or collect medical waste from the hand or gloves of the user during use of said tool. Further, unlike reusable medical device handles which must be sterilize after use and therefore teach away from creating nooks, crannies, cavities and/or wells wherein medical waste may collect, the collection handle herein is disposable and need not be sterilized for reuse.

Briefly stated, the disposable medical drill guide devices according to implementations of the present disclosure obviate the shortfalls of prior medical drill guides.

### DRAWINGS

The above-mentioned features of the present disclosure will become more apparent with reference to the following description taken in conjunction with the accompanying drawings wherein like reference numerals denote like elements and in which:

FIG. 1 shows a perspective view of some aspects of a drill guide of the disclosure;

FIG. 2 shows a partial cut-away view of some aspects of a drill guide of the disclosure;

FIGS. 3A-3J shows a cut-away view of some aspects of a drill guide of the disclosure;

FIG. 4 shows a side view of some aspects of a drill guide of the disclosure;

FIG. 5 shows a cut-away along A-A of FIG. 3; and,

FIG. 6 shows a drill guide with dual heads.

While the specification concludes with claims defining the features of the present disclosure that are regarded as novel, it is believed that the present disclosure's teachings will be better understood from a consideration of the following description in conjunction with the appendices, figures, in which like reference numerals are carried forward. All descriptions and callouts in the Figures are hereby incorporated by this reference as if fully set forth herein.

### FURTHER DESCRIPTION

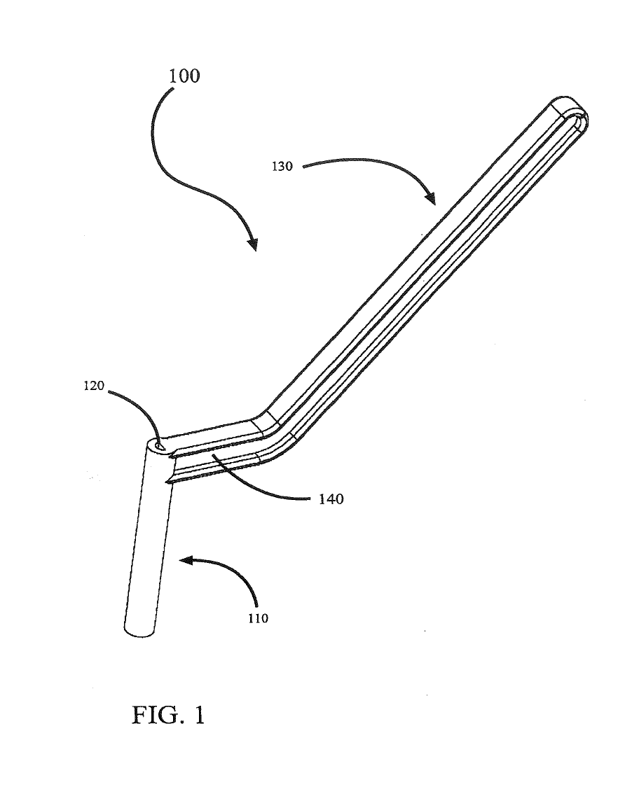

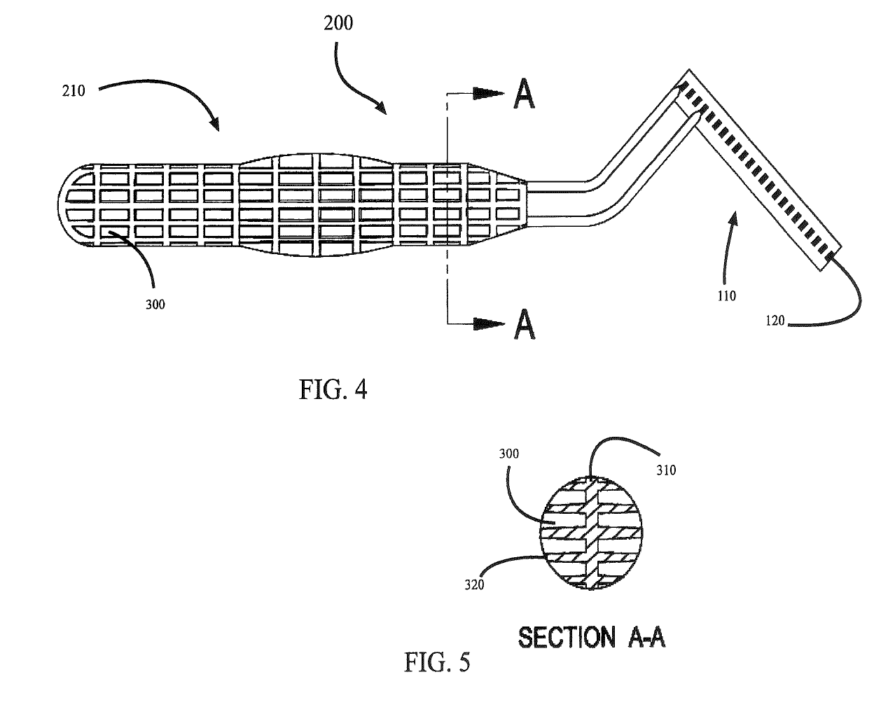

According to one or more exemplary implementations, as shown in FIGS. 1-6 medical waste disposal devices are illustrated. According to one or more exemplary implementations, as shown in FIGS. 1-band 4 disposable drill guides are illustrated.

According to aspects of one or more exemplary implementations, a disposable medical drill guide 100 formed of a non-metal structure such as plastic, polymer and/or resin with a head 110, drill channel 120 and handle 130 is taught. The head is a generally elongated hollow structure whereby a drill may be guided to a location. The head provides a pass-through for said drill.

During drilling procedures on living tissue it is important to position the drilling or other cutting instrument such as a blade or burr) precisely to avoid damaging surrounding tissue and to make the cut in the location desired.

During medical procedures that involve cutting medical waste is normally generated. Such medical waste creates problem. First it must be properly disposed of both Federal and State regulations control the disposal of medical waster. Second, said medical waste is organic, can be slippery, viscous, and gooey. Such waste can coat the handle of a medical tool to instrument thereby causing said handle to slide, rotate, move or slip in the hand of a user.

A collection zone 140 presented and formed in the side of the handle 130 provides an indented region, which may act as a container or a reservoir wherein medical waste can be collected or removed from the hand of a user. Said collection zone places the medical waste in a collection vehicle for proper disposal. The collection zone on the disposable tool provides for tool use with reduced medical waste on the tool thereby reducing the need to wipe waste off the tool handle. Accordingly, less collateral medical waste is created—said collateral waste (wiping material) also must be properly disposed of. Further, less medical waste on said handle reduces the coasting of medical waste on the handle and may reduce interference between the user's hand and the handle (also known as the hand-tool handle interface).

The head may be single component with a drill channel 120 formed of the same material as the rest of the device 100. In some instances it may be preferably to fix a sleeve 150 in said drill guide 125 by co-molding, glue, sonic weld, press fit or the like. In some instances it may be preferable to removable or changeably fit said sleeve 150 in said drill guide. Changeable fitting may include, but is not limited to, press fit, threaded, latch-catch, and friction. Said sleeve 150 may be formed of a different plastic or polymer than said handle or said head. The sleeve 150 also may be formed of a metallic substance or composite. In some instances the drill guide 125 may be a large diameter wherein it may accommodate sleeves of varying diameter thereby providing for using on handle and head to support multiple drill sleeve guides 500. The drill sleeve 150 provides the drill guide 500 that forms a positioning pathway for a drill or other cutting instrument.

In some instances one or more handle-head of a device 100 may be packaged in a kit with a palette of sleeves each of varying sizes. Said kit may be sterilized for a single use with a variety of available sleeves to match the necessary drill.

According to aspects of one or more exemplary implementations, a disposable medical drill guide 200 formed of a non-metal structure such as plastic, polymer and/or resin with a head 110, drill channel 120/125 and handle 110 is taught. The head is a generally elongated hollow structure whereby a drill may be guided to a location. The head provides a pass-through for said drill.

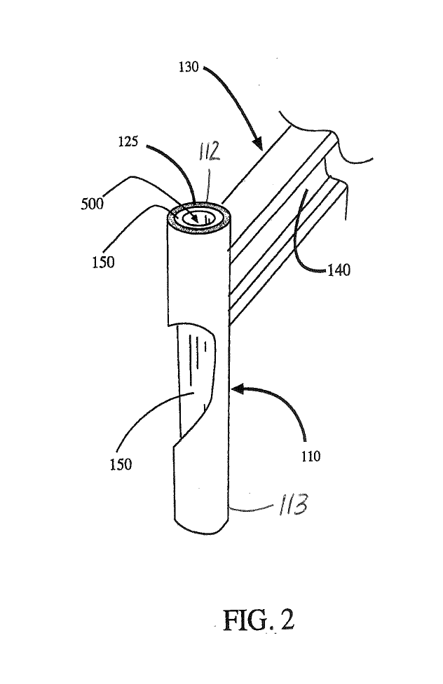

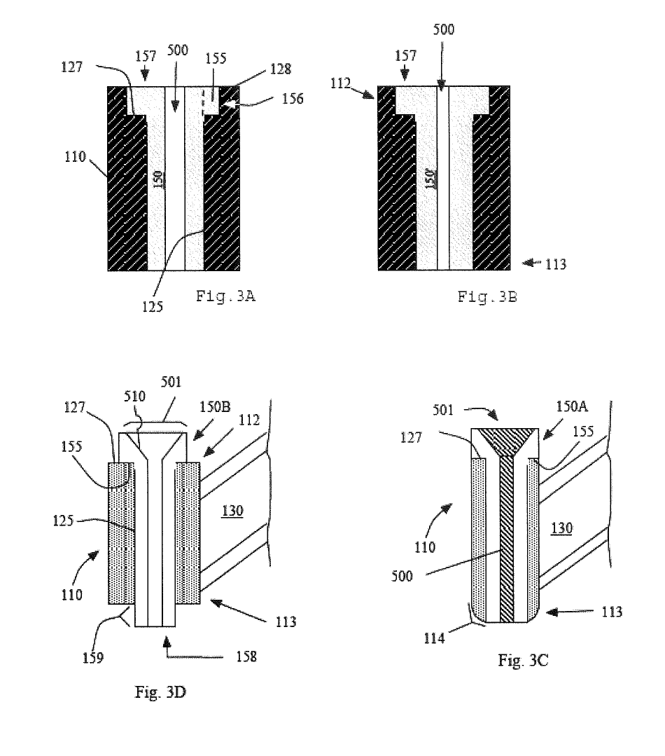

As illustrated in FIGS. 2-31 insertable metal sleeves are used in some exemplary implementations with the plastic head 110 of a drill guide device 100. The head 110 is formed as part of the handle 130 which has a medical waste collection area 140. The medical waste collection area may have wells 310 (as shown also in FIG. 5) are formed between the spine 310 of the handle and generally perpendicular ribs 320. Each sleeve 150/150′/150A-G fit, at least in part into the drill channel 125 formed in the plastic head 110. a The sleeves 150-150G each have a shoulder 155 which rests on and a shoulder 155 at the proximal end of the sleeve 156 acts as a stop against a catch 127 formed by the head 110 adjacent to the drill channel 125. The catch may also be, in some instance the top of the head 128. In other instance the catch is formed axially in the drill guide 125 but below the top of the head 128 said the sleeves may be press fit, friction fit, glued, welded, bonded, locked, threaded, screwed in or otherwise fixed or removable latched to the head. The top of a sleeve 157 may be flush with the top of the head 128, or it may rise at least partially above the top of the head 128.

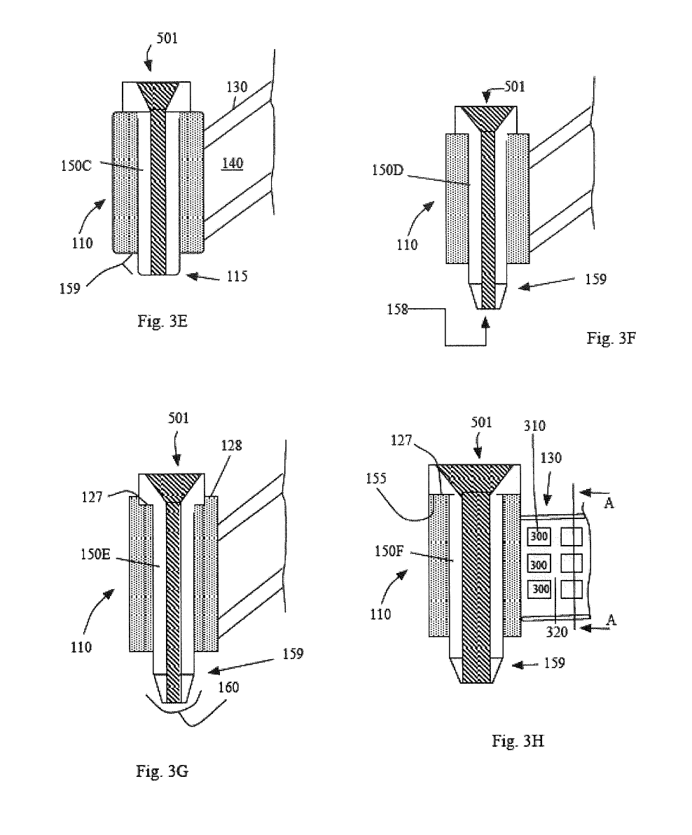

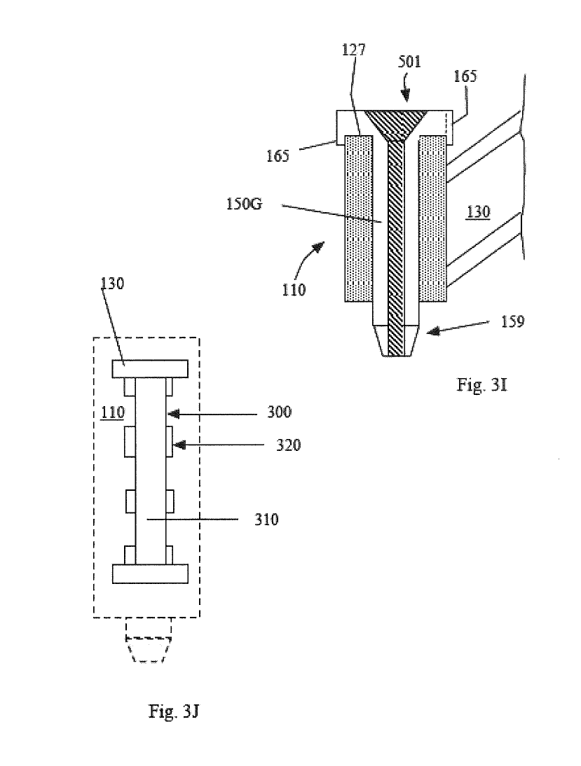

The shoulder may 155 sit above the catch 127 (FIGS. 3C-3F and 3H) above the proximal end 112 of the head. The shoulder 155 may sit partially within the head's proximal end 112 as shown in FIG. 3G. The shoulder 155 may sit within the head's proximal end 112 as shown in FIGS. 3A-3B or the head may encapsulate a part of the head's proximal end 112 as shown in FIG. 31. An extended annular wall 165 may be formed to capture a portion of the top of the head 128.

The metal region, forming the proximal end 156 of the sleeve provides an entry point 501 into the entry point 501 to the drill guide 500. The drill guide may be countersunk 510 to promote off-axis drill point entry.

In some instance the distal end 113 of the head may have a tapered portion 114. The taper assists with positioning a drill guide's dial end into an implant, fixture or plate device to align it prior to drilling.

In some instance the distal end 158 of the sleeve may have an extended portion 159 which may have a radius 115 to provide for an easier fit into a fixture or implant. In some instance the distal end 158 of the sleeve may have an extended portion 159 which has a taper 160 the taper being useful to fit the end of the drill guide into a fixture, implant, guide or other appliance for drilling into a bone.

During drilling procedures on living tissue it is important to position the drilling or other cutting instrument such as a blade or burr) precisely to avoid damaging surrounding tissue and to make the cut in the location desired.

During medical procedures that involve cutting medical waste is normally generated. Such medical waste creates problems. First it must be properly disposed of both Federal and State regulations control the disposal of medical waster. Second, said medical waste is organic, can be slippery, viscous, and gooey. Such waste can coat the handle of a medical tool to instrument thereby causing said handle to slide, rotate, move or slip in the hand of a user. By forming collection wells 300 in the handle the disclosures teaches a method of also reducing slippage or improving grip.

In FIGS. 3J-5 a series of well 300 which may also be referred to as collection zones are presented and formed in the handle 130/210. Said wells provide for collection of medical waste for or removal and disposal and to guide such waste from the hand of a user. Said wells place the medical waste in a collection vehicle for disposal. The collection zone on the disposable tool provides for tool use with reduced medical waste on the tool thereby reducing the need to wipe waste off the tool handle. Accordingly, less collateral medical waste is created—said collateral waste (wiping material) also must be properly disposed of. Further, less medical waste on said handle reduces the coating of medical waste on the handle and may reduce interference between the user's hand and the handle (also known as the hand-tool handle interface).

The head may be a single component with a drill channel formed therein or a multi element piece such as those describe in reference to FIGS. 2-3J.

The wells 300 are formed between the spine 310 of the handle and the generally perpendicular ribs 320. Those of ordinary skill in the art will recognize that a variety of spines and rib like structures forming intersecting walls can be used to form well structures, as well as annular walls, and that such structures are within this disclosure. The wells are to capture waste material, and are generally 3, 4, or 5 sided regions. Further, said walls also function as scrapers to remove waste off the hand/glove of a user.

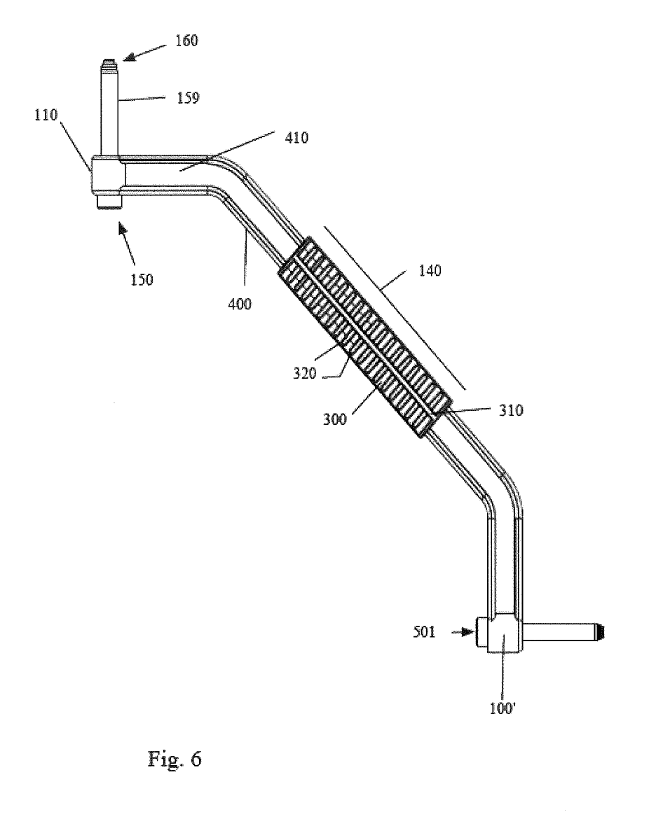

FIG. 6, shows a two sided drill guide with a first and second head 110/110′ connected by a unitary handle 400 with a waste collection area 140.

While the method and apparatus have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

It should be noted that the material and surface of the wells or collection zone(s) may be adjusted to correspond to the intended usage. A highly lubricous material may be less adequate in collecting fluid, gooey medical waste. A textured or rough surface may facilitate the collection and retention of medical waste. A combination well wall with a highly smooth and/or slick (or lubricous) upper wall (nearest the open top of a well) and a more textured or rougher region nearer the core may retain more medical waste. Walls, in some instances, may be substantially smooth. Hybrid walls or multi-zone walls may be characterized by a transition between surface features. Said multi-zones may be a bright line transition, or a more fuzzy transition. Those of ordinary skill in the art will also recognize that a multi-zone may have a plurality of wall zones of different thickness, texture, properties, geometries, etc.

1. A medical waste collection device comprising:
a head (110) having a proximal end (112), a distal end (113) and a drill channel (125) formed axially therein;
a handle;
a metallic sleeve (150) fitted into said drill channel having a drill guide (500) and a shoulder (155);
an entry point (501) to the drill guide;
at least one well formed on the handle; and,
wherein medical waste may collect in said well.

2. The device of claim 1, wherein the drill guide is countersunk (510).

3. The device of claim 1, wherein the distal end of the head is tapered (114).

4. The device of claim 1, wherein the metallic sleeve has a proximal end and a distal end.

5. The device of claim 4, wherein the distal end has radius (115).

6. The device of claim 4, wherein the metallic sleeve has a proximal end and a distal end and the distal end has an extended portion (159).

7. The device of claim 6, wherein the extended portion is tapered (160).

8. The device of claim 1 wherein the shoulder is at least partially above the proximal end of the head.

9. The device of claim 1 wherein the shoulder is above the proximal end of the head.

10. A medical waste collection device comprising:
Two heads each having a proximal end, a distal end and a drill channel formed axially therein;
An elongated handle;
a metallic sleeve (150) fitted into each of said drill channel having a drill guide and a shoulder;
an entry point to each drill guide;
at least one well formed in the handle; and,
wherein medical waste may collect in said well.

11. The device of claim 10, wherein the drill guides are countersunk.

12. The device of claim 10 wherein at least one of the shoulders is at least partially above at least one of the proximal end of at least one head.

13. The device of claim 10, wherein the metallic sleeve has a proximal end and a distal end.

14. The device of claim 13, wherein the distal end has radius (115).

15. The device of claim 13, wherein the metallic sleeve has a proximal end and a distal end and the distal end has an extended portion (159).

16. The device of claim 15, wherein the extended portion is tapered (160).
